(19) United States
(12) Patent Application Publication
(10) Pub. No.: US 2020/0163555 A1
(43) Pub. Date: May 28, 2020

(54) LIGHT SHIELDING MEMBER AND PHOTOACOUSTIC DEVICE

(71) Applicant: CANON KABUSHIKI KAISHA, Tokyo (JP)

(72) Inventors: Tomonori Ishikawa, Utsunomiya-shi (JP); Takahiro Noguchi, Tokyo (JP); Hitomi Ogasawara, Kawasaki-shi (JP)

(21) Appl. No.: 16/774,433

(22) Filed: Jan. 28, 2020

Related U.S. Application Data
(63) Continuation of application No. PCT/JP2018/020642, filed on May 30, 2018.

(30) Foreign Application Priority Data
Aug. 3, 2017 (JP) 2017-150394

(51) Int. Cl. A61B 5/00

(52) U.S. Cl. CPC A61B 5/0095; A61B 5/742

(57) ABSTRACT

A light shielding member covering a subject placement portion in which the subject is placed and a light irradiation unit that irradiates the subject with light through the subject placement portion while imaging is not underway in a photoacoustic device that images a subject and includes the subject placement portion and the light irradiation unit, the light shielding member includes a display surface that indicates positional information relating to an imaging available region on the subject.

### CROSS-REFERENCE TO RELATED APPLICATIONS

This application is a Continuation of International Patent Application No. PCT/JP2018/020642 filed on May 30, 2018 which claims the benefit of Japanese Patent Application No. 2017-150394, filed on Aug. 3, 2017, both of which are hereby incorporated by reference herein in their entirety.

### BACKGROUND OF THE INVENTION

### Field of the Invention

The present invention relates to a device that acquires subject information using the photoacoustic effect.

### Background Art

In medical fields, recent years have witnessed advances in research into the imaging of structural information and physiological information, or in other words functional information, relating to the interior of a subject. Photoacoustic tomography (PAT) has recently been proposed as one of these techniques.

When a living organism serving as a subject is irradiated with light such as laser light, and the light is absorbed by biological tissue inside the subject, an acoustic wave (typically, an ultrasonic wave) is generated. This phenomenon is known as the photoacoustic effect, while the acoustic wave generated by the photoacoustic effect is known as a photoacoustic wave. The tissue constituting the subject absorbs optical energy at different rates, and therefore the acoustic pressure of the generated photoacoustic waves also differs. In PAT, the generated photoacoustic waves are received by a probe, and by mathematically analyzing the received signals, characteristic information about the interior of the subject can be acquired.

In the X-ray diagnostic device described in PTL 1, by disposing a center mark indicating the center of a scanning region, a user is informed of the position in which to dispose the subject.

Meanwhile, in a device using photoacoustic tomography, a high-output light source such as a laser light source is used, and therefore, from the point of view of safety, an opening section is preferably closed by a light shielding member or the like during an initial stage. However, when the opening section is closed, it becomes impossible to confirm a range in which imaging can be performed, and as a result, it may become impossible to adopt an imaging posture in a correct position. Moreover, when fine position adjustments are required after adopting the imaging posture, it may be necessary to stand up in order to readopt the posture, adjust the position of a cushion, and so on, and as a result, a burden placed on an imaging technician and the subject may increase.

The present invention has been designed in consideration of these problems in the prior art, and an object thereof is to position a subject accurately in a photoacoustic device.

### CITATION LIST

### Patent Literature

[PTL 1] Japanese Patent Application Publication No. 2015-85115

### SUMMARY OF THE INVENTION

A light shielding member according to the present invention is the light shielding member covering a subject placement portion in which the subject is placed and a light irradiation unit that irradiates the subject with light through the subject placement portion while imaging is not underway in a photoacoustic device that images a subject and includes the subject placement portion and the light irradiation unit, the light shielding member includes a display surface that indicates positional information relating to an imaging available region on the subject.

Further, a photoacoustic device according to the present invention includes a subject placement portion in which a subject is placed, a light irradiation unit configured to irradiate the subject with light through the subject placement portion, an acoustic wave detector configured to detect a photoacoustic wave from the subject, a light shielding member having a display surface that indicates positional information relating to an imaging available region on the subject, and a driving unit configured to move the light shielding member to either a first position which is a position in which the light from the light irradiation unit is blocked, or a second position which is a position in which the subject is irradiated with the light from the light irradiation unit, wherein the driving unit moves the light shielding member to the first position at a timing when imaging is complete and moves the light shielding member to the second position at a timing when positioning of the subject is complete.

Further features of the present invention will become apparent from the following description of exemplary embodiments with reference to the attached drawings.

### BRIEF DESCRIPTION OF THE DRAWINGS

FIG. 1 is a view showing a configuration of a photoacoustic device according to a first embodiment.

FIG. 2 is a view illustrating an imaging posture adopted by a testee.

FIG. 3 is a view illustrating an imaging posture adopted by the testee.

FIGS. 4A and 4B are views illustrating a relationship between an indicator 101 and an imaging range.

FIGS. 5A and 5B are views illustrating the relationship between the indicator 101 and the imaging range.

FIGS. 6A and 6B are views showing a bed unit 1 according to a second embodiment.

FIG. 7 is a view showing a configuration of a photoacoustic device according to a third embodiment.

FIG. 8 is a view illustrating a problem to be solved by the present invention.

### DESCRIPTION OF THE EMBODIMENTS

Preferred embodiments of the present invention will be described below with reference to the figures. Note, however, that dimensions, materials, shapes, relative arrangements, and so on of constituent components described below are to be modified as appropriate in accordance with the configuration of the device to which the invention is applied and various conditions. Accordingly, the scope of the invention is not limited to the following description.

The present invention relates to a technique for detecting a photoacoustic wave propagating from a subject and generating and acquiring characteristic information about the interior of the subject. Accordingly, the present invention can be taken as a photoacoustic device or a control method therefor, or as a subject information acquisition method. The present invention can also be taken as a program for causing an information processing device including hardware resources such as a CPU and a memory to execute these methods, and a computer-readable, non-temporary storage medium storing the program. Furthermore, the present invention can be taken as a light shielding member used by the photoacoustic device.

The photoacoustic device according to the present invention receives an acoustic wave that is generated inside the subject when the subject is irradiated with light (electromagnetic waves), and uses the photoacoustic effect to acquire characteristic information about the subject in the form of image data. In this case, the characteristic information is information indicating characteristic values corresponding respectively to a plurality of positions inside the subject, and is generated using reception signals acquired by receiving photoacoustic waves.

The characteristic information acquired by photoacoustic measurement takes a value reflecting an absorption rate of optical energy. For example, the characteristic information includes the generation source of the acoustic wave generated in response to light irradiation, an initial acoustic pressure inside the subject, an optical energy absorption rate or absorption coefficient derived from the initial acoustic pressure, and a concentration of a tissue-forming substance.

Further, by determining an oxyhemoglobin concentration and a deoxyhemoglobin concentration as the substance concentration, an oxygen saturation distribution can be calculated. A glucose concentration, a collagen concentration, a melanin concentration, fat and water volume fractions, and so on can also be determined. Substances having a distinguishing light absorption spectrum, for example a contrast medium such as ICG (Indocyanine Green) delivered into the body, may also be used as subjects.

On the basis of the characteristic information relating to respective positions within the subject, a two-dimensional or three-dimensional characteristic information distribution is acquired. Distribution data can be generated in the form of image data. The characteristic information may be determined as distribution information relating to respective positions within the subject, rather than numerical value data. More specifically, the distribution information indicates an initial acoustic pressure distribution, an energy absorption density distribution, an absorption coefficient distribution, an oxygen saturation distribution, and so on.

The acoustic wave according to this specification is typically an ultrasonic wave, and includes elastic waves referred to as sound waves and acoustic waves. An electric signal converted from an acoustic wave using a probe or the like will also be referred to as an acoustic signal. Note, however, that the terms ultrasonic wave and acoustic wave as used in this specification are not intended to limit the wavelength of the corresponding elastic waves. An acoustic wave generated by the photoacoustic effect will be referred to as a photoacoustic wave or an optical ultrasonic wave. An electric signal derived from a photoacoustic wave will also be referred to as a photoacoustic signal. Note that in this specification, a photoacoustic signal is a concept encompassing both an analog signal and a digital signal. The distribution data will also be referred to as photoacoustic image data and reconstructed image data.

### First Embodiment

A photoacoustic device according to a first embodiment is a device that irradiates a subject with pulsed light and, by analyzing an acoustic wave generated within the subject, visualizes, or in other words forms an image of, information relating to optical characteristics of the interior of the subject. The subject is a part of the body of a testee.

<Problems in Prior Art>

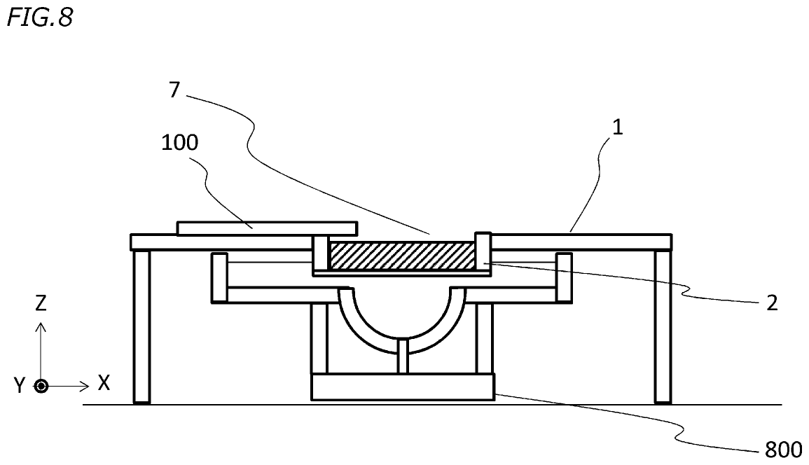

FIG. 8 is a sectional view of a photoacoustic device according to the prior art. In the example shown in FIG. 8, the testee climbs onto a bed unit 1, inserts the subject through an opening section 7 provided in a support surface, and places the subject on a holding member 2, whereupon measurement is performed. Further, the reference numeral 800 refers to a measurement unit including a light irradiation unit for irradiating the subject with light and a probe for receiving acoustic waves. The measurement unit is constituted by a hemispherical member on which the probe is disposed and a member (a matching vessel) housing a liquid (an acoustic matching liquid) used to match the acoustic inductance of the probe and the subject, and is configured so that the entire unit can move along an XY plane. By moving the probe in accordance with the matching vessel, acoustic waves can be received from a wide range while maintaining acoustic alignment with the subject.

Further, the photoacoustic device shown in FIG. 8 includes a light shielding lid 100 for closing the opening section 7. The light shielding lid is configured to be capable of sliding on the XY plane and serves to block unintended emission of laser light while imaging is not underway.

However, in a case where measurement is performed using this device, the following problem occurs.

Specifically, the range (referred to hereafter as the imaging range) in which the subject can be irradiated with light and acoustic waves can be acquired cannot be presented to the testee in advance. For example, in a case where it is discovered, after the testee adopts the measurement posture and the light shielding lid is opened, that the position of the subject is unfavorable (in a case where the subject is outside the imaging range or the like), the testee must adopt the posture again, leading to an increase in the burden placed on the testee and an assistant.

The photoacoustic device according to the first embodiment, with which this problem is solved, will be described below.

<Device Configuration>

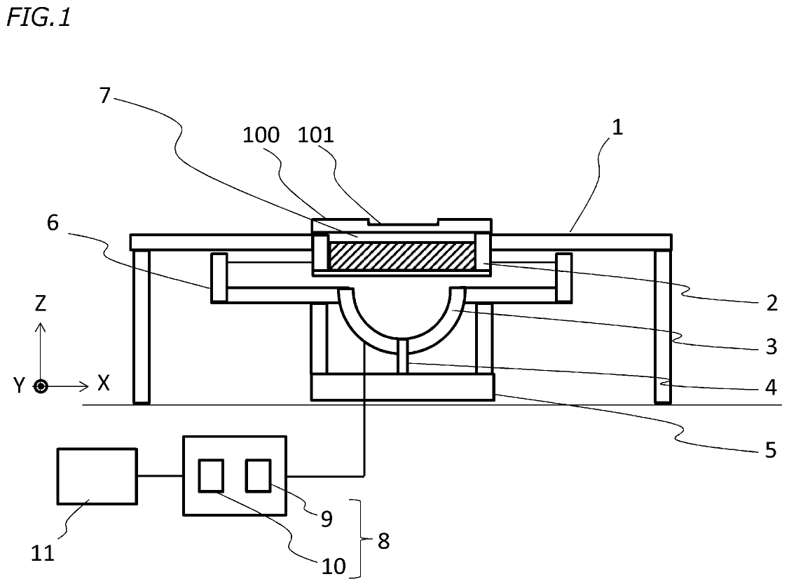

Referring to FIG. 1, the configuration of the photoacoustic device according to the first embodiment will be described.

The bed unit 1 is a unit for carrying the testee, and the testee climbs onto the bed unit 1 and is photographed while maintaining a posture. The bed unit 1 is provided with the opening section 7 into which the subject is inserted, and the holding member 2 is disposed in the opening section 7.

Further, the light shielding lid 100 that covers the opening section 7 is provided on the opening section 7 and configured to prevent light emitted from a light irradiation unit 4 (described below) from leaking to the outside while imaging is not underway.

The holding member 2 is a member (a subject carrying section) for holding the subject inserted through the opening section 7. The holding member is preferably formed from a material that transmits the light emitted from the light irradiation unit 4. For example, a mesh configuration or the like can be used.

A cap (not shown) having a high light transmittance is disposed on the holding member 2. A member having a high light transmittance (preferably at least 90%) is preferably used as the cap. Polymethylpentene, PET, polycarbonate, elastomer, and so on may be cited as specific materials. The thickness of the cap is preferably reduced in order to suppress attenuation of the acoustic waves.

Further, an acoustic matching material (a gel, water, or the like, for example) for matching the acoustic inductance of the subject and the holding member 2 is preferably housed in the holding member 2.

As long as the holding member 2 is capable of holding the subject and has a high light transmittance, the holding member 2 may be provided in a form other than mesh. For example, a sheet-form film, a rubber sheet, and so on may also be applied.

A sensor unit 3 is a unit (an acoustic wave detector) for receiving acoustic waves generated from the subject irradiated with light. The sensor unit 3 is formed from a hemispherical member, and a plurality of acoustic elements (not shown) are disposed on an inner surface thereof. The light irradiation unit 4 for emitting the light with which the subject is irradiated is provided in a bottom section of the sensor unit 3. Note that a camera for confirming the position of the subject may also be provided in the sensor unit 3.

The sensor unit 3 is placed on a driving unit 5 (described below) for moving the sensor unit in a two-dimensional direction.

The acoustic elements disposed in the sensor unit 3 are elements for receiving photoacoustic waves and converting the photoacoustic waves into electric signals. A piezoelectric ceramic material such as PZT (lead zirconium titanate), a piezoelectric polymer film material such as PVDF (polyvinylidene difluoride), and so on can be used as the members constituting the acoustic detection elements. Elements other than piezoelectric elements may also be used. For example, electrostatic capacitance type elements such as CMUTs (Capacitive Micro-machined Ultrasonic Transducers) may be used.

The light irradiation unit 4 serves as means for irradiating the subject with light and is disposed on the bottom section of the sensor unit 3. The light irradiation unit 4 is connected to a light source, and light is guided thereto through an optical system (neither the light source nor the optical system is shown in the figures).

The light source connected to the light irradiation unit 4 is a device for generating pulsed light. In order to acquire a large output, laser is preferably used as the light source, but a light emitting diode or the like may also be used. To generate a photoacoustic wave effectively, the subject must be irradiated with light for a sufficiently short period corresponding to a heat characteristic of the subject. In a case where the subject is a living organism, the pulse width of the pulsed light generated from the light source is preferably set at not more than several tens of nanoseconds. Further, the wavelength of the pulsed light is preferably approximately 700 nm to 1200 nm, which is a near-infrared region known as the biological window. Light in this region can reach comparatively deep parts of the living organism, and therefore information about the deep parts can be acquired. Note that when measurement is limited to the surface part of the living organism, a wavelength of approximately 500 to 700 nm may be used. Furthermore, the wavelength of the pulsed light preferably has a high absorption coefficient with respect to the observation subject.

The light generated by the light source is guided to the subject through an optical system. The optical system is constituted by optical devices such as lenses, mirrors, prisms, optical fibers, diffusion plates, and so on, for example. Further, while the light is being guided, these optical devices may be used to modify the shape and optical density thereof so as to realize a desired light distribution. The optical devices are not limited to those cited here, and any devices that satisfy the above functions may be used. As regards the allowable intensity of light emitted onto biological tissue, a maximum permissible exposure (MPE) is prescribed by safety standards. The maximum permissible exposure is a light intensity that can be emitted per unit surface area. Hence, by irradiating the surface of the subject with light over a large surface area at one time, a large amount of light can be guided to the subject, and as a result, a photoacoustic wave can be received at a high S/N ratio. For this reason, it is more preferable to spread the light over a certain surface area than to condense the light using a lens.

The sensor unit 3 is integrated with a matching vessel 6 holding a matching liquid for achieving acoustic alignment with the holding member 2. The matching liquid is supplied to and discharged from the matching vessel 6 by a liquid supply-discharge unit, not shown in the figure. Thus, the acoustic elements provided in the sensor unit 3 and the holding member 2 can be acoustically linked. Note that the matching liquid, similarly to the material housed in the holding member, preferably has a high transmission characteristic and a low attenuation characteristic relative to acoustic waves. Oil, water, or the like, for example, may be used favorably.

In this embodiment, the sensor unit 3, the light irradiation unit 4, and the matching vessel 6 will be referred to collectively as a measurement unit.

The driving unit 5 is a two-dimensional moving stage for moving the measurement unit in an X-Y direction. Here, the X direction is a horizontal direction on the paper surface, and the Y direction is a vertical direction on the paper surface. As long as the driving unit 5 can drive the measurement unit, any desired mechanism, such as a link mechanism, a gear mechanism, or a hydraulic mechanism, may be used. Moreover, a rotary mechanism may be used instead of linear driving using a linear guide.

A computer 8 includes a calculation section 9 and a storage section 10 and serves as means for processing electric signals output from the acoustic elements and controlling respective sites of the device such as the driving unit 5 and the light source. The computer 8 uses the electric signals output from the acoustic elements to generate an image representing characteristic information and structural information about the interior of the subject, and outputs the generated image to a monitor 11.

The calculation section 9 is typically constituted by an element such as a CPU, a GPU, or an A/D converter, or a circuit such as an FPGA or an ASIC. Note that the calculation section 9 does not have to constituted by a single element or circuit and may be constituted by a plurality of elements and circuits. Further, the processing performed by the computer 8 may be executed by any element or circuit. The storage section 10 is typically constituted by a storage medium such as a ROM, a RAM, or a hard disk. Note that the storage section 10 does not have to be constituted by a single storage medium and may be constituted by a plurality of storage media.

The calculation section 9 is capable of implementing signal processing on the electric signals output from the plurality of acoustic elements. Further, the calculation section 9 controls operations of the respective configurations constituting the photoacoustic device. Note that the computer 8 is preferably configured to be capable of performing pipeline processing simultaneously on a plurality of signals. As a result, the time required to acquire subject information can be shortened.

The processing performed by the computer 8 may be stored in the storage section 10 as a program to be executed by the calculation section 9. The storage section 10 storing the program is a non-temporary recording medium.

The monitor 11 is a device for displaying the subject information output from the computer 8 in the form of a distribution image, a numerical value in a specific region of interest, or the like. The computer 8 may include an input section on which a user inputs desired information. A keyboard, a mouse, a dial, a button, or the like, for example, may be used as the input section.

Next, an operation of the photoacoustic device according to this embodiment will be described.

The photoacoustic device according to this embodiment includes the light shielding lid 100 (a light shielding member) for protecting the eyes and so on from the light emitted from the light irradiation unit 4. The light shielding lid 100 is configured to be capable of moving to a position (a first position) covering the opening section and a position (a second position) exposing the opening section. The light shielding lid 100 remains in a closed state until imaging preparation is complete, and at a timing when imaging preparation is complete, the light shielding lid 100 is opened and the subject is inserted.

In the photoacoustic device according to this embodiment, an indicator 101 indicating position information relating to the imaging range (the imaging available region) is provided on an upper surface (a display surface) of the light shielding lid, and when the testee adopts the imaging posture on the bed unit 1, the indicator 101 informs the testee of a position with which to align the subject.

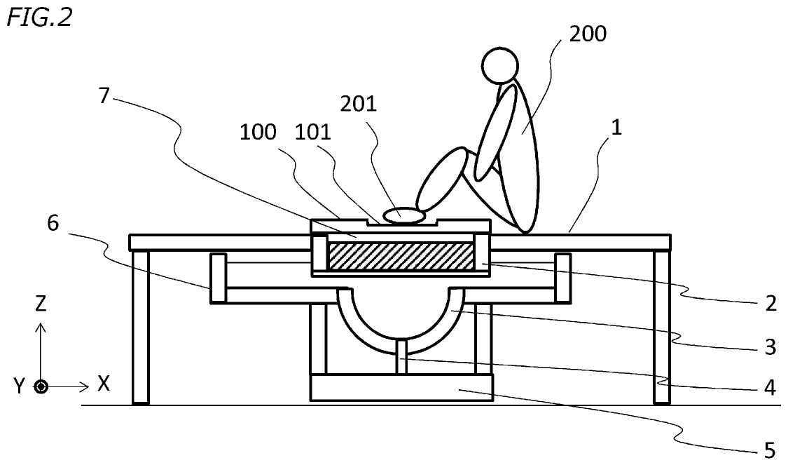

FIG. 2 is a view showing an imaging posture adopted in a case where the subject is a foot. As shown in FIG. 2, a testee 200 places a foot serving as a subject 201 in alignment with the indicator 101 provided on the light shielding lid 100, whereupon imaging preparation is performed.

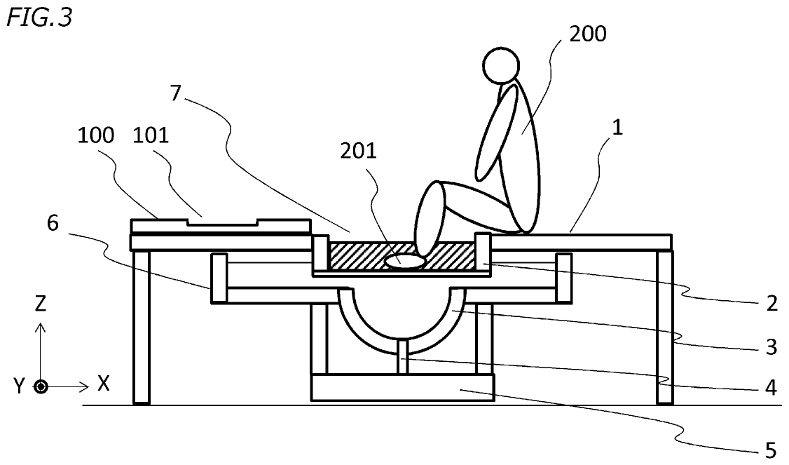

Next, as shown in FIG. 3, the light shielding lid 100 is opened, and the subject 201 is brought into contact with the holding member 2. The subject 201 has been aligned in advance with the indicator 101 indicating the imaging range, and therefore, by inserting the subject into the opening section as is, the subject can be disposed in the ideal position. Note that at this time, the position may be finely adjusted on the basis of an image captured by the camera.

The light shielding lid 100 is preferably configured so that the imaging posture of the testee 200 does not vary greatly when the light shielding lid 100 is opened. For example, the light shielding lid 100 may be configured to slide on the XY plane. Alternatively, the light shielding lid 100 may be configured so as to be placed on the opening section 7 from above.

Further, when the light shielding lid 100 has a sliding configuration, a mechanism (a rail or the like) for moving the light shielding lid 100 in parallel may be disposed on the bed unit 1. A driving unit for moving the light shielding lid 100 by motive force may also be disposed. In this case, the light shielding lid 100 may be moved using a linear actuator or the like.

Note that the sliding mechanism may be provided with a latch mechanism, not shown in the figures, so that the light shielding lid 100 can be latched in the position for adjusting the imaging posture, shown in FIG. 2. By providing the latch mechanism, the positional precision of the indicator 101 formed on the light shielding lid 100 relative to the bed unit 1 is secured. Moreover, the likelihood of the light shielding lid shifting during the procedure for positioning the subject can be reduced. The latch mechanism can be provided on an end of the sliding region in alignment with the aforementioned first position of the light shielding lid 100. Similarly, the latch mechanism cam be provided in alignment with the second position.

Further, the driving unit may be configured to implement control for opening the light shielding lid 100 at a timing when imaging preparation is complete (for example, a timing when positioning of the subject 201 is complete or a timing when the testee finishes adopting the imaging posture). The driving unit may also be configured to implement control for closing the light shielding lid 100 at a timing when imaging is complete. The light shielding lid 100 may be opened and closed on the basis of input performed by the user or on the basis of a sensing result acquired in relation to the testee. For example, the light shielding lid may be opened at a timing when an imaging start operation is performed or opened automatically after sensing, at the start of imaging, that the position of the subject and the posture of the testee are stable. Further, the light shielding lid may be closed at a timing when imaging is complete and the testee dismounts.

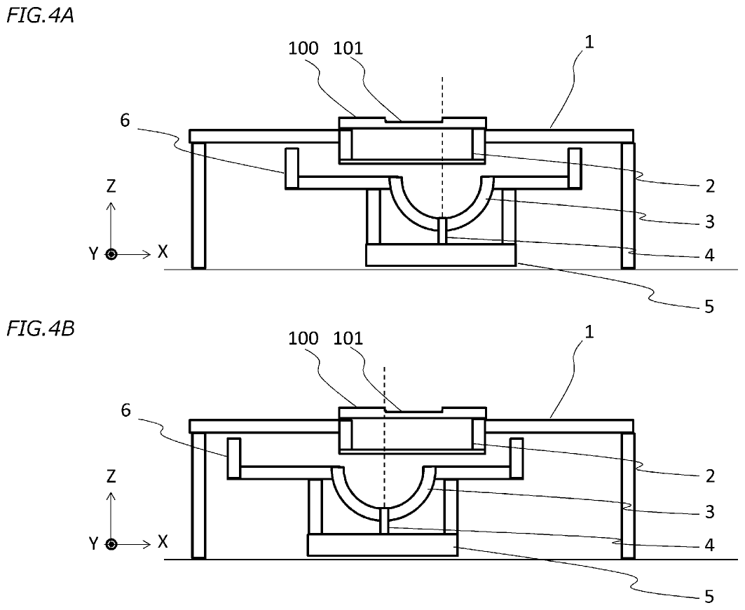

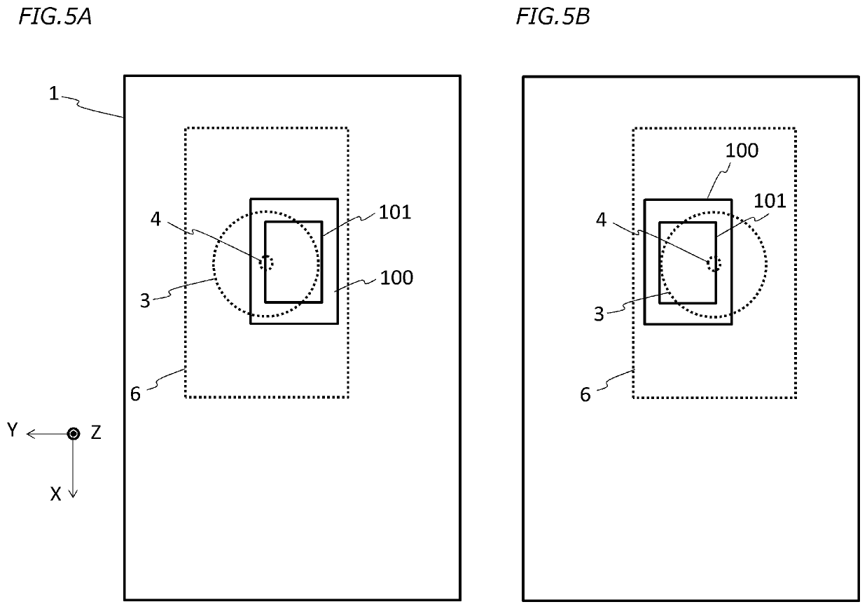

FIGS. 4A, 4B, 5A, and 5B are views showing a relationship between the indicator 101 and the imaging range.

Dotted lines shown respectively in FIGS. 4A and 4B represent the center of the light emitted from the light irradiation unit 4. FIG. 4A shows a state in which the driving unit 5 has been moved by a maximum extent in an X axis positive direction, and FIG. 4B shows a state in which the driving unit 5 has been moved by a maximum extent in an X axis negative direction. The imaging range of the photoacoustic device in the X axis direction is between the illustrated dotted lines.

Further, FIG. 5A shows a state in which the driving unit 5 has been moved by a maximum extent in a Y axis positive direction, and FIG. 5B shows a state in which the driving unit 5 has been moved by a maximum extent in a Y axis negative direction. The imaging range of the photoacoustic device in the Y axis direction is within the movable range of the light irradiation unit 4.

Hence, in this embodiment, a rectangle indicated by the indicator 101 represents the movable range of the light irradiation unit 4. This range matches the range (the range in which imaging can be performed) in which the subject information can be formed into an image.

Note that in this example, the indicator 101 is a step (a depressed portion) provided in the upper surface of the light shielding lid 100, but the indicator 101 may take another form. For example, the indicator 101 may indicate the imaging range by lines or may be a mark or the like indicating the center of the imaging range. Alternatively, a surface that impinges on the subject 201 may be provided.

In a case where the indicator 101 is not provided on the light shielding lid 100, depending on the posture adopted by the testee 200, the subject 201 may deviate from the imaging range. Moreover, in this case, it becomes necessary to open the light shielding lid 100 and adopt the posture again, leading to an increase in the burden on the testee and the assistant. In particular, when the photoacoustic device has the form shown in the figures, the holding member 2 is filled with the acoustic matching material (water or the like), and therefore water may splash up when the subject moves. It may also become necessary to rearrange a cushion or the like.

The photoacoustic device according to this embodiment, however, is capable of informing the testee 200 of the imaging range in a state where the light shielding lid 100 is closed, and therefore the correct imaging posture can be adopted from the start, thereby lightening the burden on the testee and the assistant.

### Second Embodiment

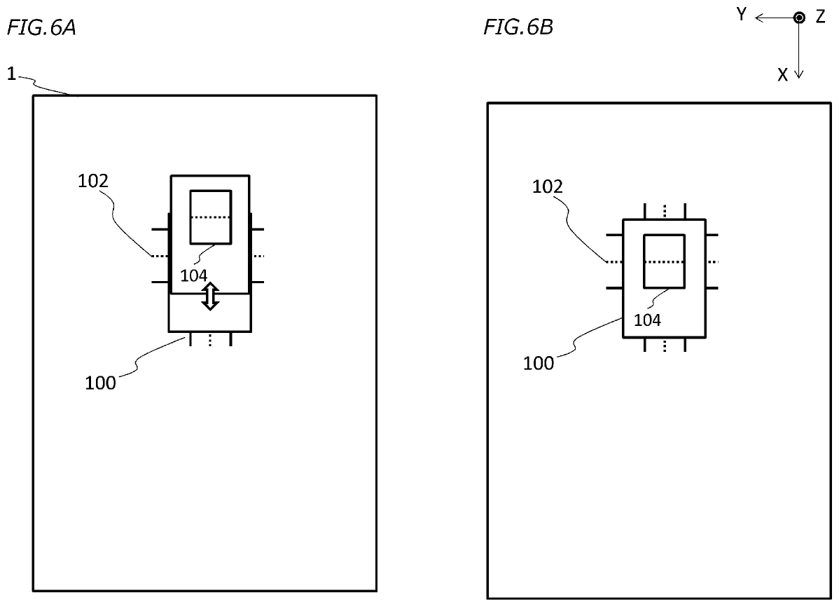

In the first embodiment, the indicator 101 is provided on the upper surface of the light shielding lid 100, but the indicator may be provided on the bed unit 1 side. FIGS. 6A and 6B are views showing the bed unit 1 from a Z axis positive direction. In a second embodiment, a scale 102 serving as a marker indicating the imaging range is provided on the bed unit 1 on the periphery of the opening section 7.

In the second embodiment, the position in which to latch the light shielding lid 100 can be specified using both a scale 104 provided on the light shielding lid 100 and the scale 102 provided on the bed unit.

FIG. 6A shows a state in which the light shielding lid 100 is open (a state in which positional deviation has occurred relative to the bed unit 1), and FIG. 6B shows a state in which the light shielding lid 100 is closed (in other words, a state in which the subject can be positioned). Note that the scale 102 shown in FIGS. 6A and 6B indicates the edges (solid lines) of the imaging range and the center (dotted line) of the imaging range, but other positional relationships may be illustrated by the scale 102.

### Third Embodiment

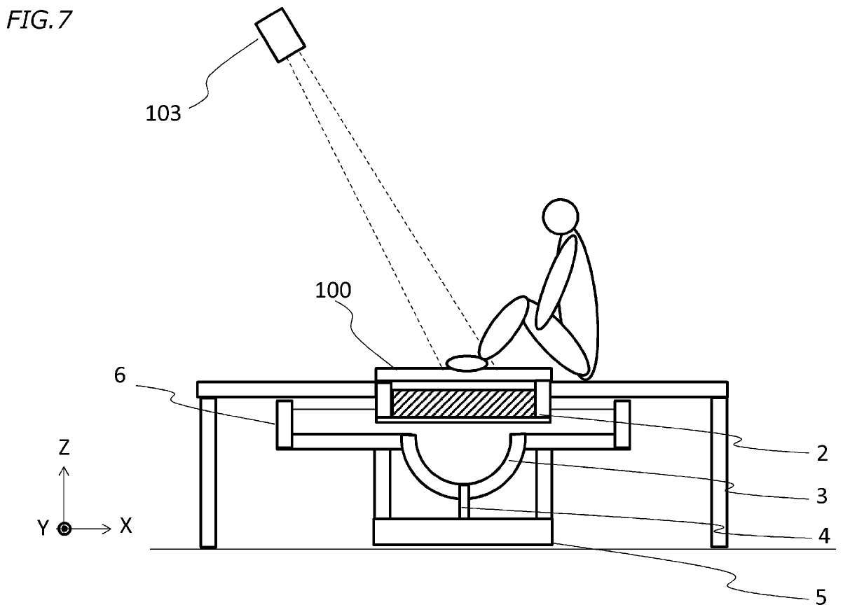

In the first embodiment, the indicator 101 is provided physically on the upper surface of the light shielding lid 100, but the indicator may be formed by being optically projected. As shown in FIG. 7, for example, a projector 103 may be provided in the room where the device is disposed, and the indicator may be projected onto the upper surface of the light shielding lid 100. In this case, the upper surface of the light shielding lid 100 is preferably formed from a material onto which light can be projected. By projecting the indicator 101, the position with which to align the subject can be indicated regardless of the position of the light shielding lid 100, and therefore positioning precision can be secured.

Further, a display device (for example, a light emitting diode, a liquid crystal display, or the like) may be provided on the upper surface of the light shielding lid 100, and the indicator may be displayed thereon. By making the indicator displayable, the testee can be provided with various information.

According to the present invention, a subject can be positioned accurately in a photoacoustic device.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A light shielding member covering a subject placement portion in which the subject is placed and a light irradiation unit that irradiates the subject with light through the subject placement portion while imaging is not underway in a photoacoustic device that images a subject and includes the subject placement portion and the light irradiation unit, the light shielding member comprises:
a display surface that indicates positional information relating to an imaging available region on the subject.

2. The light shielding member according to claim 1, wherein
the positional information is information expressing the imaging available region in a case where the subject is placed in the subject placement portion.

3. The light shielding member according to claim 2, wherein
a portion indicating the positional information is provided on the display surface.

4. The light shielding member according to claim 2, wherein
the display surface is a surface having an optical characteristic enabling projection of the positional information thereon.

5. The light shielding member according to claim 2 further comprises:
a display device capable of displaying the positional information on the display surface.

6. The light shielding member according to claim 1, wherein
the light shielding member is capable of moving to a first position covering the subject placement portion and the light irradiation unit and a second position exposing the subject placement portion and the light irradiation unit.

7. The light shielding member according to claim 6, wherein
the first position is a position in which the light from the light irradiation unit is blocked, and
the second position is a position in which the subject is irradiated with the light from the light irradiation unit.

8. The light shielding member according to claim 6, wherein,
in the first position, the positional information indicates the imaging available region in a case where the subject is placed in the subject placement portion.

9. A photoacoustic device comprising:
a light shielding member according to claim 1;
the subject placement portion;
the light irradiation unit; and
an acoustic wave detector configured to detect a photoacoustic wave from the subject.

10. The photoacoustic device according to claim 9, further comprising:
a projecting unit configured to project the positional information onto the display surface.

11. The photoacoustic device according to claim 9, further comprising:
a driving unit configured to move the light shielding member to the first position covering the subject placement portion and the light irradiation unit and the second position exposing the subject placement portion and the light irradiation unit.

12. A photoacoustic device comprising:
a subject placement portion in which a subject is placed;
a light irradiation unit configured to irradiate the subject with light through the subject placement portion;
an acoustic wave detector configured to detect a photoacoustic wave from the subject;
a light shielding member having a display surface that indicates positional information relating to an imaging available region on the subject; and
a driving unit configured to move the light shielding member to either a first position which is a position in which the light from the light irradiation unit is blocked, or a second position which is a position in which the subject is irradiated with the light from the light irradiation unit, wherein
the driving unit moves the light shielding member to the first position at a timing when imaging is complete and moves the light shielding member to the second position at a timing when positioning of the subject is complete.
